(19) United States
(12) Patent Application Publication
(10) Pub. No.: US 2012/0196281 A1
(43) Pub. Date: Aug. 2, 2012

(54) FUNCTIONALIZED ORGANOTYPIC SYSTEMS

(76) Inventors: Volker Busskamp, Newton, MA (US); Jens Duebel, Wurzburg (DE); Serge Picaud, Avon (FR); Botond Roska, Oberwil (CH); José Alain Sahel, Paris (FR)

(21) Appl. No.: 13/498,619

(22) PCT Filed: Sep. 28, 2010
(86) PCT No.: PCT/EP2010/064325

§ 371 Date: Apr. 18, 2012

(30) Foreign Application Priority Data
Sep. 29, 2009 (EP) 09171574.8

(51) Int. Cl. C12Q 1/68; C12Q 1/02; C12N 5/02

(52) U.S. Cl. 435/6.1; 435/375; 435/29

(57) ABSTRACT

The present invention provides an organotypic system comprising a portion of a retina containing live cells in a cultured in medium, wherein photosensitivity has been restored in at least some of said live cells. Methods of use thereof are also provided.

### FIELD OF THE INVENTION

The present invention relates to an organotypic system comprising a portion of a retina containing live cells in a cultured in medium, wherein photosensitivity has been restored in at least some of said live cells.

### BACKGROUND OF THE INVENTION

Whole animal experiments are laborious, expensive, and time consuming to perform, and should be circumvented if possible. In addition, relatively large amounts of test compound must be synthesized in order to dose animals. For example, under current animal testing protocols, a minimum of 7 animals/data point is generally required due to variation in animals and high sensitivity required of the assays. Each determination in organotypic culture requires a fraction of the number of animals as a similar determination in vivo, e.g. approximately 3 slices per data point, vs. 7 animals/data point. Moreover, dose response and time course studies performed in organotypic system experiments facilitate better initial choices for in vivo dosing regimens, reducing the number of in vivo experiments with adjusted dosing regiments required. Another major drawback of whole animal experiments in the number of variables which cannot be controlled and are difficult to assess. For example, if a compound is without effect, it may be due to rapid clearance from the blood, rapid metabolism, sequestration by a non-target tissue, or inability to penetrate the blood brain barrier. Dosing may be limited by toxicity to a sensitive non-target organ. Determining the contribution of these factors to a negative result is a major undertaking. Thus, negative results are not of use in generating structure-activity relationships to guide generation of improved compound structures. Organotypic culture systems eliminate or minimize these variables since e.g. the blood brain barrier and other tissues are not present. Metabolism of compound is easily assessed by sampling media. Dose at the target organ is easily controlled.

Thus there is a need in the art to develop systems for the study of neurodegenerative disorders, vision restoration, drug discovery, tumor therapies and diagnosis of disorders.

### SUMMARY OF THE INVENTION

At the time of the invention, it was known that retina could be prepared and kept in culture for a short amount of time. However, it had been observed that for unknown reasons the degenerate extremely quickly and hence loose their primary function, i.e. detection of light. As a consequence, the cultured retinas were used to study their architecture.

The present inventors have now surprisingly found that it is possible to restore photosensitivity in cultured retina in such a way that functional outputs, i.e. light dependent outputs can be measured. Cultured retina of the invention thus provide a solution to the above problem in that they can be used as experimental models for e.g. drug screening, or study of disease development.

The present invention hence provides an organotypic system comprising a portion of a retina containing live cells in a cultured in medium, wherein photosensitivity has been restored in at least some of said live cells. In some embodiments, cells of the organotypic system of the invention are viable for at least two weeks in culture in the absence of a toxic agent.

In some embodiments, the organotypic system of the invention comprises a photosensitive molecule which has been targeted to at least one live cell of said portion of a retina. In some embodiments, the targeting is done using a vector targeted to cellular components of the retina, said vector expressing a photosensitive molecule in some viable cells. Where a targeting vector is used, the photosensitive molecule can be a channelrhodopsin or a halorhodopsin. Photoswitchable affinity label can also be applied without the need to use a vector.

In some embodiments, the photosensitive molecule has been targeted to, or is expressed in, a photoreceptor, such as a cone or a rod, a bi-polar cell, for instance an On bi-polar cell or an Off bi-polar cell, an amacrine cell, such as an On amacrine cell, an Off amacrine cell or an On-Off amacrine cell, a ganglion, for example an On ganglion, an Off ganglion or an On-Off ganglion, a horizontal cell or a glia cell.

In some embodiments, the vector is targeted to a cellular component of a retina, said vector expressing at least one reporter gene which is detectable in living cells. Such reporter genes can be indicative of a functioning neural circuit. Examples of such vectors are activity sensors or a rainbow viruses.

Viral vectors, for instance an AAV, a PRV or a lentivirus, are suitable to target and deliver genes to cells of the organotypic system of the invention.

The output of the organotypic system of the invention can be measured using an electrical method, such as a multi-electrode array or a patch-clamp, or using a visual method, such as the detection of fluorescence.

In addition, diseases, such as macular degeneration, can induced and studied in the organotypic systems of the invention.

In some embodiments of the invention, the retina is a human retina.

The organotypic system of the invention can be used in a method for identifying therapeutic agents for the treatment of a neurological disorder or of a disorder of the retina, said method comprising the steps of contacting a test compound with an organotypic system of the invention, and comparing at least one output obtained in the presence of said test compound with the same output obtained in the absence of said test compound.

Moreover, the organotypic system of the invention can also be used in a method for in vitro testing of vision restoration, said method comprising the steps of contacting an organotypic system of the invention with an agent, and comparing at least one output obtained after the contact with said agent with the same output obtained before said contact with said agent.

### BRIEF DESCRIPTION OF THE DRAWINGS

FIG. 1: Lentiviral construct. Lentiviral eNpHR-EYFP expression construct, SIN=self inactivating, LTR=long tandem repeat, Psi=extended packaging signal, cPPT=central polypurine tract.

FIG. 2: Transfected human photoreceptors. Confocal top view on immunostained eNpHR-EYFP expressing human photoreceptors; reconstructed side projection, eNpHR-EYFP staining; co-stained with DAPI; scale bar=20 μm.

FIG. 3: Tile scan of transfected area. Confocal top view of the transfected area of a 7d incubated human retinal tissue 2 days post lentiviral administration, scale bar=250 μm.

FIG. 4: eNpHR-mediated current/voltage responses. current (left upper panel) and voltage (left lower panel) responses of an eNpHR-EYFP transfected human photoreceptor stimulated at different wavelengths; Right panel: voltage responses of a human photoreceptor stimulated at different frequencies at 580 nm.

FIG. 5: AAV-transfected human retina. Cross section of a human retina transfected with AAV2-mCAR-eNpHR-EYFP, serotype 8, left eNpHR-EYFP staining, right costained with DAPI, scale bar=20 μm.

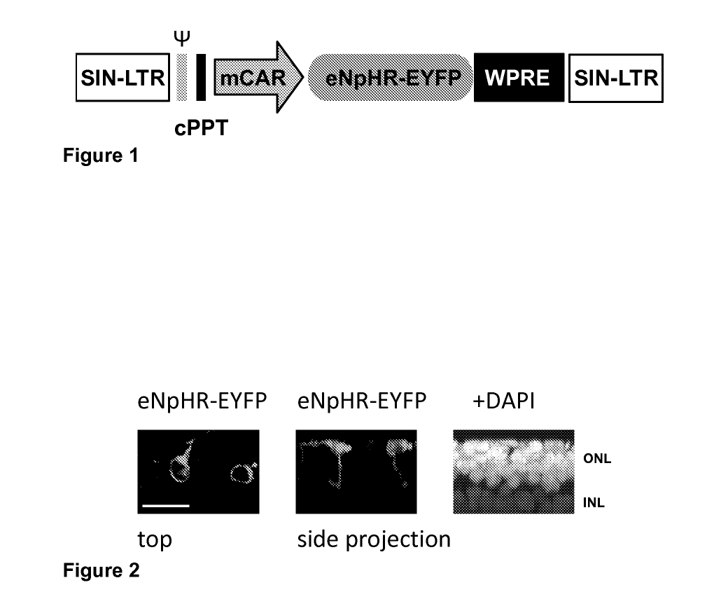

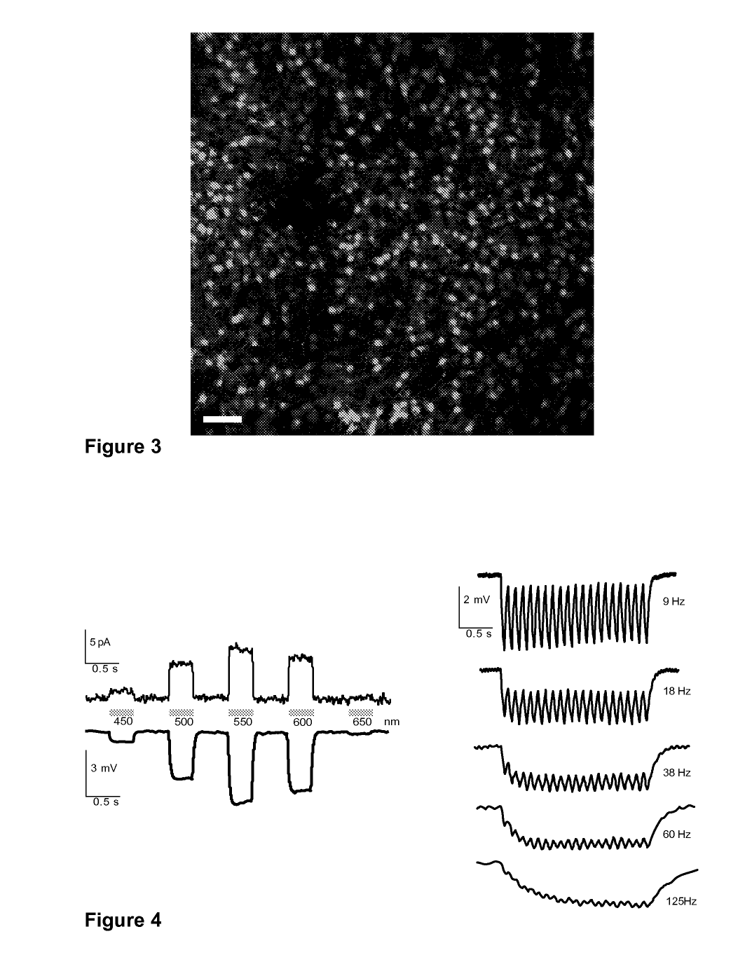

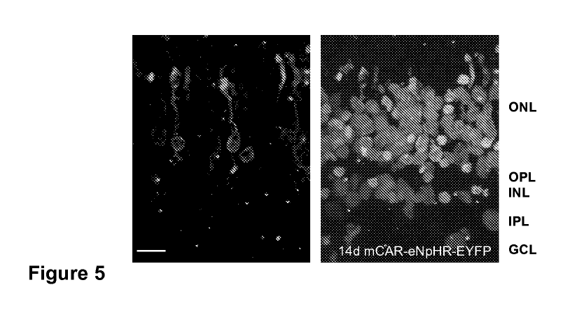

### DETAILED DESCRIPTION OF THE INVENTION

At the time of the invention, it was known that retina could be prepared and kept in culture for a short amount of time. However, it had been observed that for unknown reasons the degenerate extremely quickly and hence loose their primary function, i.e. detection of light. As a consequence, the cultured retinas were used to study their architecture.

The present inventors have now surprisingly found that it is possible to restore photosensitivity in cultured retina in such a way that functional outputs, i.e. light dependent outputs can be measured. Cultured retina of the invention thus provide a solution to the above problem in that they can be used as experimental models for e.g. drug screening, or study of disease development. This is of out most importance since the retina is also to be seen as a part of the brain. The systems of the invention thus allow an extensive and physiological study not only of the retina, but neuronal circuitry in general.

The present invention hence provides an organotypic system comprising a portion of a retina containing live cells in a cultured in medium, wherein photosensitivity has been restored in at least some of said live cells. In some embodiments, cells of the organotypic system of the invention are viable for at least two weeks in culture in the absence of a toxic agent. In some embodiments, said cells are viable for at least 3 weeks, four weeks, five weeks, six weeks, seven weeks, eight weeks, nine weeks or ten weeks.

In some embodiments, the organotypic system of the invention comprises a photosensitive molecule which has been targeted to at least one live cell of said portion of a retina. In some embodiments, the targeting is done using a vector targeted to cellular components of the retina, said vector expressing a photosensitive molecule in some viable cells. Where a targeting vector is used, the photosensitive molecule can a channelrhodopsin, a halorhodopsin. Photoswitchable affinity label can also be applied without the need to use a vector.

Channelrhodopsins are a subfamily of opsin proteins that function as light-gated ion channels. They serve as sensory photoreceptors in unicellular green algae, controlling phototaxis, i.e. movement in response to light. Expressed in cells of other organisms, they enable the use of light to control intracellular acidity, calcium influx, electrical excitability, and other cellular processes. Three channelrhodopsins are currently known: Channelrhodopsin-1 (ChR1), Channelrhodopsin-2 (ChR2), and Volvox Channelrhodopsin (VChR1). Moreover, some modified/improved versions of these proteins als oexist. All known Channelrhodopsins are unspecific cation channels, conducting H+, Na+, K+, and Ca2+ ions. Halorhodopsin is a light-driven ion pump, specific for chloride ions, and found in phylogenetically ancient “bacteria” (archaea), known as halobacteria. It is a seven-transmembrane protein of the retinylidene protein family, homologous to the light-driven proton pump bacteriorhodopsin, and similar in tertiary structure (but not primary sequence structure) to vertebrate rhodopsins, the pigments that sense light in the retina. Halorhodopsin also shares sequence similarity to channelrhodopsin, a light-driven ion channel. Halorhodopsin contains the essential light-isomerizable vitamin A derivative all-trans-retinal. Halorhodopsin is one of the few membrane proteins whose crystal structure is known. Halorhodopsin isoforms can be found in multiple species of halobacteria, including H. salinarum, and N. pharaonis. Much ongoing research is exploring these differences, and using them to parse apart the photocycle and pump properties. After bacteriorhodopsin, halorhodopsin may be the best type I (microbial) opsin studied. Peak absorbance of the halorhodopsin retinal complex is about 570 nm. Recently, halorhodopsin has become a tool in optogenetics. Just as the blue-light activated ion channel channelrhodopsin-2 opens up the ability to activate excitable cells (such as neurons, muscle cells, pancreatic cells, and immune cells) with brief pulses of blue light, halorhodopsin opens up the ability to silence excitable cells with brief pulses of yellow light. Thus halorhodopsin and channelrhodopsin together enable multiple-color optical activation, silencing, and desynchronization of neural activity, creating a powerful neuroengineering toolbox.

Photoswitchable affinity labels (PAL) consist of a ligand, a tether containing the photoswitch, and a reactive group, and specifically target endogenous ion channels. Illumination permanently modifies the ion channels on the cell surface with the PAL molecule in a way that subsequent light treatment can repetitively switch the channels between a functional and disabled state. See also Nature Methods—5, 293-295 (2008) for a review.

In some embodiments, the photosensitive molecule has been targeted to, or is expressed in, a photoreceptor, such as a cone or a rod, a bi-polar cell, for instance an On bi-polar cell or an Off bi-polar cell, an amacrine cell, such as an On amacrine cell, an Off amacrine cell or an On-Off amacrine cell, a ganglion, for example an On ganglion, an Off ganglion or an On-Off ganglion, a horizontal cell or a glia cell.

In some embodiments, the vector is targeted to a cellular component of a retina, said vector expressing at least one reporter gene which is detectable in living cells. Such reporter genes can be indicative of a functioning neural circuit. Examples of such vectors are activity sensors or rainbow viruses (Nature Methods 6, 127-130 (2009)). Examples of such viruses are retrograde, transsynaptic pseudorabies viruses (PRVs) with genetically encoded activity sensors that optically report the activity of connected neurons among spatially intermingled neurons in the brain. Such activity sensor can be an isolated transsynaptic virus expressing an exogenous fluorescent activity sensor. The transsynaptic virus can be a rhabdovirus, e.g. rabies virus, or a herpesvirus, for isntance an alphaherpesvirus, e.g. pseudorabies virus. The fluorescent exogenous activity sensor can be a fluorescent protein Ca²⁺ sensor, e.g. yellow cameleon, camgaroo, G-CaMP/Pericam, or TN-L15, or a fluorescent protein voltage sensor, e.g. FlaSh, SPARC, or a VSP, preferably VSP1.

Suitable viral vectors for the invention are well-known in the art. For instance an AAV, a PRV or a lentivirus, are suitable to target and deliver genes to cells of the organotypic system of the invention. Optimal viral delivery for photoreceptors can be achieved by mounting the ganglion cell side downwards, so that the photoreceptor side of the retina is exposed and can thus be better transfected. Another technique is slicing, e.g. with a razor blade, the inner limiting membrane of the retina, such that the delivering viruses can penetrate the inner membranes. A further way is to embed the retina in agar, slicing said retina and applying the delivery viruses from the side of the sl ice.

The output of the organotypic system of the invention can be measured using well-known methods, for instance using an electrical method, such as a multi-electrode array or a patch-clamp, or using a visual method, such as the detection of fluorescence. In some embodiments, the inner limiting membrane is remove by micro-surgery the inner limiting membrane. In other embodiments, recording is achieved through slices performed to the inner limiting membrane.

In addition, diseases, such as macular degeneration, can induced and studied in the organotypic systems of the invention. Other disease which could be induced and studied in the organotypic systems of the invention comprise photoreceptor degeneration (as occurs in, e.g., hereditary or acquired retinitis pigmentosa, cone dystrophies, cone-rod and/or rod-cone dystrophies, and macular degeneration, including age-related and early onset macular degeneration); retinal detachment and retinal trauma; photic lesions caused by laser or sunlight; a macular hole; a macular edema; night blindness and color blindness; ischemic retinopathy as caused by diabetes or vascular occlusion; retinopathy due to prematurity/premature birth; infectious conditions, such as, e.g., CMV (cytomegalovirus) retinitis, herpes type 1 retinitis, Ebstein-Barr virus retinitis, toxoplasmosis, rubella and pox virus; inflammatory conditions, such as the uveitidies, multifocal choroiditis and uveitis, birdshot chorioretinopathy, collagen vascular diseases affecting the posterior segment of the eye, including Wegener's granulomatosis, uveitis associated with systemic lupus erythematosus, uveitis associated with polyarteritis nodosa, peripheral or intermediate uveitis, chronic central serous chorioretinopathy, and myopic choroidal neovascular membranes and scars. Inflammatory disorders also include Bechet syndrome, intermediate uveitis (pars planitis), masquerade syndromes, peripheral uveitis, ocular syphilis, ocular tuberculosis, viral-related chorioretinitis (ARN) syndrome, HIV-related uveitis, progressive outer retinal necrosis syndrome, sympathetic ophthalmia, white dot syndromes, presumed ocular histoplasmosis syndrome, acute macular neuroretinopathy, diffuse unilateral subacute neuroretinitis, ophthalmomyiasis, serpiginous choroidopathy, panuveitis, birdshot retinochoroidopathy, and uveitis associated with disorders such as juvenile rheumatoid arthritis, Kawasaki syndrome, multiple sclerosis, sarcoidosis, toxocariasis, toxoplasmosis, Vogt-Koyanagi-Harada (VKH), and HLA-B27 seropositive spondylopathy syndromes. Other disorders include tumors, such as retinoblastoma and ocular melanoma. The systems of the invention can also be used to study optic nerve diseases such as optic atrophy, ischemic optic neuropathy, diabetes induced optic atrophy, optic nerve hypoplasia, morning glory syndrome, Graves ophthalmopathy, optic neuritis, cytomegalovirus neuritis, arteritic optic neuropathy, compressive neuropathy, diabetic neuropathy, giant cell arteritis, infiltrative neuropathy, nutriotional, ischemic neuropathy, retrobulbar optic neuritis, retrobulbar ischemic neuropathy, toxic neuropathy, traumatic neuropathy; optic nerve diseases resulting from causes such as syphilis, Lyme disease, toxoplasmosis, cat scratch disease, systemic lupus erythematosus, paraneoplastic syndrome, multiple sclerosis, and autoimmune disease; degenerative optic diseases such as age-related macular degeneration, early onset macular degeneration, Usher Syndrome, retinitis pigmentosa, cone-road dystrophy, and choroideremia; and congenital optical diseases such as Leber's congential amaurosis, congential stationary night blindness, and optic nerve hypoplasia. One of skill in the art will recognize that there is overlap between the various classifications of the disorders and conditions listed herein.

Any source of retina can be used for the present invention. In some embodiments of the invention, the retina is a human retina. In other embodiments, the retina is of bovine or of rodent origin. Human retina can be easily obtained from cornea banks where said retina are normally discarded after the dissection of the cornea. Adult human retina has a large surface (about 1100 mm²) and can therefore be easily separated to a number of experimentally subregions. Moreover, the resensitized retina of the invention can also be used as an exquisite model for synaptic communication since the retina has synapses that are identical to the rest of the brain.

The organotypic system of the invention can be used in a method for identifying therapeutic agents for the treatment of a neurological disorder or of a disorder of the retina, said method comprising the steps of contacting a test compound with an organotypic system of the invention, and comparing at least one output obtained in the presence of said test compound with the same output obtained in the absence of said test compound.

The organotypic system of the invention allows for the identification of tumor agents and candidate therapeutic compounds that are low in toxicity and high in efficacy. It can be combined with high through put robotic, computer, and combinatorial library screening assays. The initial screen can be carried out by the high throughput screening assay. Examples of such assays are known to one of skill in the art. Selected candidate drugs are then used in the organotypic system of the invention to identify those compounds which are therapeutic candidates or pose serious threats to an animal, such as for example, toxins, poisons and the like, especially those that affect the neural system and/or vision. A high throughput screening system for identifying therapeutic agents for treatment of neurological disorders and/or vision restoration comprises identifying a library of candidate compounds by robotic, computer screening and/or combinatorial libraries; and, contacting a test compound with a test organotypic system and measuring at least one output. Preferably, the candidate drugs are identified by laser desorption/ionization mass spectrometry, HPLC, ELISA, MALDI, SELEX, biochips or immunochemical assays.

Moreover, the organotypic system of the invention can also be used in a method for in vitro testing of vision restoration, said method comprising the steps of contacting an organotypic system of the invention with an agent, and comparing at least one output obtained after the contact with said agent with the same output obtained before said contact with said agent.

Agents identified using the methods of the invention are also encompassed within the scope of the present invention.

As used herein, the term “organotypic” refers to a tissue, removed from an organ, that continues to develop as it would in that organ. An organotypic system resembles an organ in vivo in three dimensional form or function or both.

The term “animal” is used herein to include all animals. In some embodiments of the invention, the non-human animal is a vertebrate. Examples of animals are human, mice, rats, cows, pigs, horses, chickens, ducks, geese, cats, dogs, etc. The term “ animal” also includes an individual animal in all stages of development, including embryonic and fetal stages. A “genetically-modified animal” is any animal containing one or more cells bearing genetic information altered or received, directly or indirectly, by deliberate genetic manipulation at a sub-cellular level, such as by targeted recombination, microinjection or infection with recombinant virus. The term “genetically-modified animal” is not intended to encompass classical crossbreeding or in vitro fertilization, but rather is meant to encompass animals in which one or more cells are altered by, or receive, a recombinant DNA molecule. This recombinant DNA molecule may be specifically targeted to a defined genetic locus, may be randomly integrated within a chromosome, or it may be extrachromosomally replicating DNA. The term “germ-line genetically-modified animal” refers to a genetically-modified animal in which the genetic alteration or genetic information was introduced into germline cells, thereby conferring the ability to transfer the genetic information to its offspring. If such offspring in fact possess some or all of that alteration or genetic information, they are genetically-modified animals as well.

The alteration or genetic information may be foreign to the species of animal to which the recipient belongs, or foreign only to the particular individual recipient, or may be genetic information already possessed by the recipient. In the last case, the altered or introduced gene may be expressed differently than the native gene, or not expressed at all.

The genes used for altering a target gene may be obtained by a wide variety of techniques that include, but are not limited to, isolation from genomic sources, preparation of cDNAs from isolated mRNA templates, direct synthesis, or a combination thereof.

A type of target cells for transgene introduction is the ES cells. ES cells may be obtained from pre-implantation embryos cultured in vitro and fused with embryos (Evans et al. (1981), Nature 292:154-156; Bradley et al. (1984), Nature 309:255-258; Gossler et al. (1986), Proc. Natl. Acad. Sci. USA 83:9065-9069; Robertson et al. (1986), Nature 322:445-448; Wood et al. (1993), Proc. Natl. Acad. Sci. USA 90:4582-4584). Transgenes can be efficiently introduced into the ES cells by standard techniques such as DNA transfection using electroporation or by retrovirus-mediated transduction. The resultant transformed ES cells can thereafter be combined with morulas by aggregation or injected into blastocysts from a non-human animal. The introduced ES cells thereafter colonize the embryo and contribute to the germline of the resulting chimeric animal (Jaenisch (1988), Science 240:1468-1474). The use of gene-targeted ES cells in the generation of gene-targeted genetically-modified mice was described 1987 (Thomas et al. (1987), Cell 51:503-512) and is reviewed elsewhere (Frohman et al. (1989), Cell 56:145-147; Capecchi (1989), Trends in Genet. 5:70-76; Baribault et al. (1989), Mol. Biol. Med. 6:481-492; Wagner (1990), EMBO J. 9:3025-3032; Bradley et al. (1992), Bio/Technology 10:534-539).

Techniques are available to inactivate or alter any genetic region to any mutation desired by using targeted homologous recombination to insert specific changes into chromosomal alleles.

As used herein, a “targeted gene” is a DNA sequence introduced into the germline of a non-human animal by way of human intervention, including but not limited to, the methods described herein. The targeted genes of the invention include DNA sequences which are designed to specifically alter cognate endogenous alleles.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g. , a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

“Polynucleotides” can be composed of single-and double-stranded DNA, DNA that is a mixture of single-and double-stranded regions, single-and double-stranded RNA, and RNA that is mixture of single-and double-stranded regions, hybrid molecules comprising DNA and RNA that may be single-stranded or, more typically, double-stranded or a mixture of single-and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence. “Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5× Denhardt's solution, 10% dextran sulfate, and 20 g/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of forrnamide result in lowered stringency); salt conditions, or temperature. For example, moderately high stringency conditions include an overnight incubation at 37 degree C. in a solution comprising 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment,” “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, denivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization , selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al. , Meth Enzymol 182:626-646 (1990); Rattan et al., Ann NY Acad Sci 663:48-62 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue.

“Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence aligmnent, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty—1, Joining Penalty—30, Randomization Group Length=0, Cutoff Score=I, Gap Penalty—5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty—I, Joining Penalty=20, Randomization Group Length=0, Cutoff Score=l, Window Size=sequence length, Gap Penalty—5, Gap Size Penalty—0.05, Window Size=500 or the length of the subject amino acid sequence, whichever is shorter. If the subject sequence is shorter than the query sequence due to N-or C-terminal deletions, not because of internal deletions, a manual correction must be made to the results. This is because the FASTDB program does not account for N-and C-terminal truncations of the subject sequence when calculating global percent identity. For subject sequences truncated at the N-and C-termini, relative to the query sequence, the percent identity is corrected by calculating the number of residues of the query sequence that are N-and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N-and C-termini of the subject sequence, which are not matched/aligned with the query sequence, are considered for the purposes of manually adjusting the percent identity score. That is, only query residue positions outside the farthest N-and C-terminal residues of the subject sequence. Only residue positions outside the N-and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having hepanin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N-or C-terminal residues of a protein retains such immunogenic activities can readily be determined by routine methods described herein and otherwise known in the art.

“Fusion proteins”, also known as chimeric proteins, are proteins created through the joining of two or more genes which originally coded for separate proteins. Translation of this fusion gene results in a single polypeptide with functional properties derived from each of the original proteins. Recombinant fusion proteins are created artificially by recombinant DNA technology for use in biological research or therapeutics. The functionality of fusion proteins is made possible by the fact that many protein functional domains are modular. In other words, the linear portion of a polypeptide which corresponds to a given domain, such as a tyrosine kinase domain, may be removed from the rest of the protein without destroying its intrinsic enzymatic capability. A recombinant fusion protein is a protein created through genetic engineering of a fusion gene. This typically involves removing the stop codon from a cDNA sequence coding for the first protein (first partner), then appending the cDNA sequence of the second protein (second partner) in frame through ligation or overlap extension PCR. That DNA sequence will then be expressed by a cell as a single protein. The protein can be engineered to include the full sequence of both original proteins, or only a portion of either. If the two entities are proteins, linker (or “spacer) peptides can be added.

“Label” refers to agents that are capable of providing a detectable signal, either directly or through interaction with one or more additional members of a signal producing system. Labels that are directly detectable and may find use in the invention include fluorescent labels. Specific fluorophores include fluorescein, rhodamine, BODIPY, cyanine dyes and the like.

A “fluorescent label” refers to any label with the ability to emit light of a certain wavelength when activated by light of another wavelength.

“Fluorescence” refers to any detectable characteristic of a fluorescent signal, including intensity, spectrum, wavelength, intracellular distribution, etc.

“Detecting” fluorescence refers to assessing the fluorescence of a cell using qualitative or quantitative methods. In some of the embodiments of the present invention, fluorescence will be detected in a qualitative manner. In other words, either the fluorescent marker is present, indicating that the recombinant fusion protein is expressed, or not. For other instances, the fluorescence can be determined using quantitative means, e. g., measuring the fluorescence intensity, spectrum, or intracellular distribution, allowing the statistical comparison of values obtained under different conditions. The level can also be determined using qualitative methods, such as the visual analysis and comparison by a human of multiple samples, e. g., samples detected using a fluorescent microscope or other optical detector (e. g., image analysis system, etc.) An “alteration” or “modulation” in fluorescence refers to any detectable difference in the intensity, intracellular distribution, spectrum, wavelength, or other aspect of fluorescence under a particular condition as compared to another condition. For example, an “alteration” or “modulation” is detected quantitatively, and the difference is a statistically significant difference. Any “alterations” or “modulations” in fluorescence can be detected using standard instrumentation, such as a fluorescent microscope, CCD, or any other fluorescent detector, and can be detected using an automated system, such as the integrated systems, or can reflect a subjective detection of an alteration by a human observer.

The “green fluorescent protein” (GFP) is a protein, composed of 238 amino acids (26.9 kDa), originally isolated from the jellyfish Aequorea victoria/Aequorea aequorea/Aequorea forskalea that fluoresces green when exposed to blue light. The GFP from A. victoria has a major excitation peak at a wavelength of 395 nm and a minor one at 475 nm. Its emission peak is at 509 nm which is in the lower green portion of the visible spectrum. The GFP from the sea pansy (Renilla reniformis) has a single major excitation peak at 498 nm. Due to the potential for widespread usage and the evolving needs of researchers, many different mutants of GFP have been engineered. The first major improvement was a single point mutation (S65T) reported in 1995 in Nature by Roger Tsien. This mutation dramatically improved the spectral characteristics of GFP, resulting in increased fluorescence, photostablility and a shift of the major excitation peak to 488 nm with the peak emission kept at 509 nm. The addition of the 37° C. folding efficiency (F64L) point mutant to this scaffold yielded enhanced GFP (EGFP). EGFP has an extinction coefficient (denoted c), also known as its optical cross section of 9.13×10-21 m²/molecule, also quoted as 55,000 L/(mol·cm). Superfolder GFP, a series of mutations that allow GFP to rapidly fold and mature even when fused to poorly folding peptides, was reported in 2006.

The “yellow fluorescent protein” (YFP) is a genetic mutant of green fluorescent protein, derived from Aequorea victoria. Its excitation peak is 514 nm and its emission peak is 527 nm.

As used herein, the singular forms “a”, “an,” and “the” include plural reference unless the context clearly dictates otherwise.

A “virus” is a sub-microscopic infectious agent that is unable to grow or reproduce outside a host cell. Each viral particle, or virion, consists of genetic material, DNA or RNA, within a protective protein coat called a capsid. The capsid shape varies from simple helical and icosahedral (polyhedral or near-spherical) forms, to more complex structures with tails or an envelope. Viruses infect cellular life forms and are grouped into animal, plant and bacterial types, according to the type of host infected.

The term “transsynaptic virus” as used herein refers to viruses able to migrate from one neurone to another connecting neurone through a synapse. Examples of such transsynaptic virus are rhabodiviruses, e.g. rabies virus, and alphaherpesviruses, e.g. pseudorabies or herpes simplex virus. The term “transsynaptic virus” as used herein also encompasses viral sub-units having by themselves the capacity to migrate from one neurone to another connecting neurone through a synapse and biological vectors, such as modified viruses, incorporating such a sub-unit and demonstrating a capability of migrating from one neurone to another connecting neurone through a synapse.

Transsynaptic migration can be either anterograde or retrograde. During a retrograde migration, a virus will travel from a postsynaptic neuron to a presynaptic one. Accordingly, during anterograde migration, a virus will travel from a presynaptic neuron to a postsynaptic one.

Homologs refer to proteins that share a common ancestor. Analogs do not share a common ancestor, but have some functional (rather than structural) similarity that causes them to be included in a class (e.g. trypsin like serine proteinases and subtilisin's are clearly not related—their structures out side the active site are completely different, but they have virtually geometrically identical active sites and thus are considered an example of convergent evolution to analogs).

There are two subclasses of homologs—orthologs and paralogs. Orthologs are the same gene (e.g. cytochome ‘c’), in different species. Two genes in the same organism cannot be orthologs. Paralogs are the results of gene duplication (e.g. hemoglobin beta and delta). If two genes/proteins are homologous and in the same organism, they are paralogs.

As used herein, the term “disorder refers to an ailment, disease, illness, clinical condition, or pathological condition.

As used herein, the term “pharmaceutically acceptable carrier refers to a carrier medium that does not interfere with the effectiveness of the biological activity of the active ingredient, is chemically inert, and is not toxic to the patient to whom it is administered.

As used herein, the term “pharmaceutically acceptable derivative” refers to any homolog, analog, or fragment of an agent, e.g. identified using a method of screening of the invention, that is relatively non-toxic to the subject.

The term “therapeutic agent” refers to any molecule, compound, or treatment, that assists in the prevention or treatment of disorders, or complications of disorders.

Compositions comprising such an agent formulated in a compatible pharmaceutical carrier may be prepared, packaged, and labeled for treatment.

If the complex is water-soluble, then it may be formulated in an appropriate buffer, for example, phosphate buffered saline or other physiologically compatible solutions.

Alternatively, if the resulting complex has poor solubility in aqueous solvents, then it may be formulated with a non-ionic surfactant such as Tween, or polyethylene glycol. Thus, the compounds and their physiologically acceptable solvates may be formulated for administration by inhalation or insufflation (either through the mouth or the nose) or oral, buccal, parenteral, rectal administration or, in the case of tumors, directly injected into a solid tumor.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e. g., sorbitol syrup, cellulose derivatives or hydrogenated edible fats); emulsifying agents (e. g., lecithin or acacia); non-aqueous vehicles (e. g., almond oil, oily esters, or fractionated vegetable oils); and preservatives (e. g., methyl or propyl-p-hydroxybenzoates or sorbic acid). The pharmaceutical compositions may take the form of, for example, tablets or capsules prepared by conventional means with pharmaceutically acceptable excipients such as binding agents (e. g., pregelatinized maize starch, polyvinyl pyrrolidone or hydroxypropyl methylcellulose); fillers (e. g., lactose, microcrystalline cellulose or calcium hydrogen phosphate); lubricants (e. g., magnesium stearate, talc or silica); disintegrants (e. g., potato starch or sodium starch glycolate); or wetting agents (e. g., sodium lauryl sulphate). The tablets may be coated by methods well-known in the art.

Preparations for oral administration may be suitably formulated to give controlled release of the active compound.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e. g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e. g., gelatin for use in an inhaler or insufflator may be formulated containing a powder mix of the compound and a suitable powder base such as lactose or starch.

The compounds may be formulated for parenteral administration by injection, e. g., by bolus injection or continuous infusion. Formulations for injection may be presented in unit dosage form, e. g., in ampoules or in multi-dose containers, with an added preservative.

The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e. g., sterile pyrogen-free water, before use.

The compounds may also be formulated in rectal compositions such as suppositories or retention enemas, e. g., containing conventional suppository bases such as cocoa butter or other glycerides.

The compounds may also be formulated as a topical application, such as a cream or lotion.

In addition to the formulations described previously, the compounds may also be formulated as a depot preparation. Such long acting formulations may be administered by implantation (for example, subcutaneously or intramuscularly) or by intramuscular injection.

Thus, for example, the compounds may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers therapeutically or prophylactically effective amounts of the compositions in pharmaceutically acceptable form.

The composition in a vial of a kit may be in the form of a pharmaceutically acceptable solution, e. g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e. g., saline, dextrose solution, etc.), preferably sterile, to reconstitute the complex to form a solution for injection purposes.

In another embodiment, a kit further comprises a needle or syringe, preferably packaged in sterile form, for injecting the complex, and/or a packaged alcohol pad. Instructions are optionally included for administration of compositions by a clinician or by the patient.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### EXAMPLES

Human Ocular Tissue.

Human ocular tissue was obtained from the Cornea Bank of Amsterdam and was handled in accordance with the guidelines of the Declaration of Helsinki. Eyes were collected from five donors (ranging in age from 58 to 65 years) subsequent to death through natural causes. After removal of the cornea, eyes were stored within 6 to 12 hours in cold phosphate buffered saline solution (PBS) and shipped to our laboratory. Post-mortem delay times were always less than 36 hours before explants preparation.

Retinal Explants Culture and Vector Administration. Immediately after receipt of the eyes, anterior parts were removed and vitreous humour with attached neural retina was transferred in CO₂-independant medium (Invitrogen). Retina was carefully separated from vitreous humour and medium fragments (˜1 cm²) were dissected. With the photoreceptor face up, these retinal fragments were placed on polycarbonate membrane of a Transwell 0.4 μm cell culture insert (Corning) with one drop of CO₂-independant medium and carefully flattened with a polished Pasteur pipette. CO₂-independant medium was removed and 2 ml of Neurobasal-A medium supplemented with 2 mM L-glutamine and B27 supplement were added per well (NBA+; Invitrogen). Retinal explants were exposed 2E+07 VG/mL (lentiviral genomes) for 24-72 hours. 10 to 50 μL of AAV particles (3.08E+12 to 5.33E+12 GC/mL) were dropped on top of retinal explants and incubated up to 18 days. Culture medium was renewed by a daily addition of fresh NBA+ to each well.

Light Stimulation.

Halorhodopsin (eNpHR) was stimulated with light generated by a 120 W epifluorescent mercury lamp-based illuminator (X-Cite 120PC, EXFO Photonics Solutions Inc, Canada) filtered with a band-pass filter (600-660 nm, Chroma Technology Corp., USA) and projected to the retina. The epifluorescent light path was computer controlled with a shutter (SC10, Thorlabs Ltd., UK). Light intensity was modulated by neutral density filters over a range of 5 log units (ND 0-ND 40). For pattern light stimuli photoreceptors were stimulated using light generated by a digital light projector (V-332, PLUS Vision Corp, Japan) filtered with the same band-pass filter used for the epifluorescent light path. A monochromatic light source (15 nm bandwith) was used to provide a ramp of light with a wavelength range from 400 to 700 nm or light pulses with a fixed wavelength (TILL Photonics Polychrome V, Agilent Technologies Inc). Light intensities from all light sources were measured using a fluorescent spectrometer (USB 4000, Ocean Optics Inc, USA) calibrated with a radiometric reference source (LS1-Cal, Ocean Optics). The stimulus was generated and the environment was monitored via custom-made software (Matlab, Mathworks Inc.; Labview, National Instruments).

Two-Photon Targeted Patch Clamp Recordings.

AAV infected retinas from Cnga3−/−, Rho−/− double knockout and Pde6brdl mice were isolated in Ringer's medium (in mM: 110 NaCl, 2.5 KCl, 1 CaCl₂, 1.6 MgCl₂, 10 D-glucose, 22 NaHCO₃, bubbled with 5% CO₂/95% O₂). For photoreceptor patch clamp recordings retinal slices (200 μm) were cut with a razor blade tissue chopper (Stoelting, Wood Dale, Ill., USA) and placed in the recording chamber of the microscope. The tissue was superfused in Ringer's medium at 36° C. for the duration of the experiment. AAV infected fluorescent photoreceptors were targeted with a patch electrode by using a custom made 2-photon microscope which allowed the visualization of the retina with a CCD camera during two-photon scanning. Whole-cell recordings were made using an Axon Multiclamp 700B amplifier. Patch electrodes were made from borosilicate glass (BF100-50-10, Sutter Instruments) pulled to 8-10 MΩ, and filled with (in mM): 115 K Gluconate, 10 KCl, 1 MgCl₂, 0.5 CaCl2, 1.5 EGTA, 10 HEPES, 4 ATP-Na₂, (pH 7.2). For ganglion cell recordings the retina was mounted ganglion cell side up on a filter paper (MF-membrane, Millipore, USA) with a 2 mm opening in the middle. Ganglion cell spike recordings were performed with loose cell-attached patch, using the same electrodes pulled to 6-8 MΩ, and filled with Ringer's medium. Whole-cell recordings were made with electrodes filled with (in mM): 115 K Gluconate, 10 KCl, 1 MgCl₂, 0.5 CaCl₂, 1.5 EGTA, 10 HEPES, 4 ATP-Na₂, 7.5 neurobiotin chloride (pH 7.2). Excitatory and inhibitory currents were isolated by voltage clamping ganglion cells at the reversal potential of Cl⁻ (−60 mV) and non-selective cation channels (0-20 mV). For these recordings electrodes were filled with (in mM): 112.5 CsMeSO₄, 1 Mg SO₄, 7.8×10-3 CaCl₂, 0.5 BAPTA, 10 HEPES, 4 ATP-Na₂, 0.5 GTP-Na₃, 5 lidocaine N-ethyl bromide (QX314-Br), 7.5 neurobiotin chloride (pH 7.2). For human photoreceptor patch clamp recordings we used human retinal explants which were incubated with the lentivirus for 1-2 days. The human retinas were cut into slices (200 μm) with a razor blade tissue chopper using the same method as described above. Brightly labeled photoreceptors from the parafoveal region were targeted with a patch electrode (8-10 MΩ) filled with (in mM): 115 K Gluconate, 10 KCl, 1 MgCl₂, 0.5 CaCl₂, 1.5 EGTA, 10 HEPES, 4 ATP-Na₂, (pH 7.2). Patch-clamp recordings were also made from non-infected human photoreceptors immediately after the isolation of the retina in order to prove the absence of photocurrents in untreated cultured human retinas. The human tissue was superfused in Ringer's medium at 36° C. for the duration of the patch-clamp experiment. All chemicals were obtained from Sigma, with the exception of ATP (Labforce) and neurobiotin (Vector Laboratories). Data was analyzed offline with Matlab R15 (Mathworks Inc.).

Multi-Electrode Array Recordings.

To record the spike trains of retinal ganglion cells, the isolated mouse retina was placed on a flat MEA60 200 Pt GND array with 30 μm diameter microelectrodes spacing 200 μm (Ayanda Biosystems, Lausanne, Switzerland). The retina was continuously superfused in oxygenated Ringer's solution (110 mM NaCl, 2.5 mM KCl, 1.0 mM CaCl₂, 1.6 mM MgCl₂, 22 mM NaHCO₃, 10 mM D-glucose (pH 7.4 with 95% O₂ and 5% CO₂)) at 37° C. during experiments. Recordings ranged from 1-5 h in duration. The signals were recorded (MEA1060-2-BC, Multi-Channel Systems, Germany) and filtered between 500 Hz (low cut-off) and 3500 Hz (high cut-off). The spikes were extracted with a threshold of 4 times the standard deviation of the recorded trace (Matlab; Mathworks Inc.).

Statistical Analysis.

The statistical significance was determined using a one-tailed heteroscedastic Student's t-test if n >7 and the Lilliefors test did not reject the hypothesis that the sample have normal distribution at 5% significance level else the Wilcoxon rank-sum test was used. Comparing groups, we used the Kruskal-Wallis test to perform a non-parametric 1-way ANOVA. The different levels of significance are indicated by * if p<0.05, ** if p<0.01, and *** if p<0.001. The error bars and ± values represent s.e.m.

Using a lentiviral vector, the present inventors found high levels of eNpHR expression specifically in human photoreceptors even after only 1-2 days of incubation. Brightly labeled photoreceptors in the parafoveal region displayed large photocurrents and photovoltages with spectral tuning reflecting eNpHR activation. Human photoreceptor voltage was modulated with periodic light stimulation up to 60 Hz, a smaller value than in mice, most probably due to the additional capacitance of the intact outer segments in human photoreceptors. The present inventors did not measure any photocurrents from control human retinas; even at the time of the isolation of the retina.

The present inventors have found that a single microbial gene restored complex responses, mediated via the endogenous ON and OFF channels in human retina, thus restoring their photosensitivity. Their finding that human photoreceptors can be specifically transduced, together with a recent report that dysfunctional cone photoreceptors were still alive more than 30 years after disease onset in a retinitis pigmentosa patient, suggest a potential for translating eNpHR-based functional rescue of visual function to humans. The action spectrum of eNpHR, with a peak at 580 nm, is advantageous because exposure to light at lower wavelengths leads to more tissue damage. Furthermore, the resensitized retinal culture of the invention will pave the way to investigate physiologically-stimulated human neural circuit and synaptic function at single cell resolution, as well as for screening and testing compounds that affect molecular pathways in health and disease in an approachable part of the human brain.

1. An organotypic system comprising a portion of a retina containing live cells in a cultured in medium, wherein photosensitivity has been restored in at least some of said live cells.

2. The organotypic system of claim 1, wherein the portion of a retina containing live cells is viable for at least two weeks in culture in the absence of a toxic agent.

3. The organotypic system of claim 1, wherein a photosensitive molecule has been targeted to at least one live cell of said portion of a retina.

4. The organotypic system of claim 1, wherein a vector has been targeted to cellular components of said portion of a retina, said vector expressing a photosensitive molecule in some viable cells.

5. The organotypic system of claim 1, wherein the photosensitive molecule is a channelrhodopsin, a halorhodopsin or a photoswitchable affinity label.

6. The organotypic system of claim 1, wherein a photosensitive molecule has been targeted to, or is expressed in, a photoreceptor, such as a cone or a rod, a bi-polar cell, for instance an On bi-polar cell or an Off bi-polar cell, an amacrine cell, such as an On amacrine cell, an Off amacrine cell or an On-Off amacrine cell, a ganglion, for example an On ganglion, an Off ganglion or an On-Off ganglion, a horizontal cell or a glia cell.

7. The organotypic system of claim 1, wherein a vector has been targeted to a cellular component of said portion of a retina, said vector expressing at least one reporter gene which is detectable in living cells.

8. The organotypic system of claim 7, wherein detection of reporter genes is indicative of a functioning neural circuit, and wherein said vector is an activity sensor or a rainbow virus.

9. The organotypic system of claim 1, wherein the vector is a viral vector.

10. The organotypic system of claim 1, wherein the output of the retina cells is measured using an electrical method, or using a visual method.

11. The organotypic system of claim 1, wherein a disease has been induced in said system.

12. The organotypic system of claim 1, wherein the retina is a human retina.

13. A method for identifying therapeutic agents for the treatment of a neurological disorder or of a disorder of the retina, said method comprising the steps of contacting a test compound with an organotypic system of claim 1, and comparing at least one output obtained in the presence of said test compound with the same output obtained in the absence of said test compound.

14. A method for in vitro testing of vision restoration, said method comprising the steps of contacting an organotypic system of claim 1 with an agent, and comparing at least one output obtained after the contact with said agent with the same output obtained before said contact with said agent.

15. The organotypic system of claim 9, wherein the viral vector is an AAV, a PRV, or a lentivirus.

16. The organotypic system of claim 10, wherein the electrical method is a multi-electrode array or a patch-clamp method and wherein the visual method is detection of fluorescence.

17. The organotypic system of claim 11, wherein the disease is macular degeneration.
